(19) United States
(12) Patent Application Publication
(10) Pub. No.: US 2024/0306945 A1
(43) Pub. Date: Sep. 19, 2024

(54) ADAPTIVE LOUDNESS SCALING

(71) Applicant: Cochlear Limited, Macquarie University, NSW (AU)

(72) Inventor: Stefan LIEVENS, Herent (BE)

(21) Appl. No.: 18/262,288

(22) PCT Filed: Jan. 3, 2022
(86) PCT No.: PCT/IB2022/050025

§ 371 Date: Jul. 20, 2023

Related U.S. Application Data
(60) Provisional application No. 63/142,754, filed on Jan. 28, 2021.

(51) Int. Cl. A61B 5/12; A61B 5/00

(52) U.S. Cl. CPC A61B 5/123; A61B 5/7435

(57) ABSTRACT

During a first set of loudness or intensity level scaling tests, a first set of response options are provided to a user of a sensory prosthesis, and the first set of response options are used to receive indications of the user's perceived loudness of the delivered stimulation signals. The indications of a perceived loudness of the stimulation signals, received via the first set of response options, are used to adapt the first set of response options to a second set of response options for use during a second set of loudness or intensity level scaling tests. The first set of response options are associated with a first response precision level, while second set of response options are associated with a second response precision level that is different from the first response precision level.

### BACKGROUND

### Field of the Invention

The present invention relates generally to hearing devices.

### Related Art

Medical devices have provided a wide range of therapeutic benefits to users over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or user monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a user. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: performing one or more first loudness scaling tests during which sounds are delivered to an ear of a user via a hearing device; during the one or more first loudness scaling tests, providing the user with a first set of response options; receiving, via the first set of response options, indications of the user's perceived loudness of the sounds delivered to the user during the one or more first loudness scaling tests; and based on the indications of the user's perceived loudness of the sounds delivered to the user during the one or more first loudness scaling tests, adapting the first set of response options to a second set of response options for use during one or more second loudness scaling tests.

In another aspect, one or more non-transitory computer readable storage media are provided. The one or more non-transitory computer readable storage media comprise instructions that, when executed by at least one processor, are operable to: display, via a display screen, a first set of loudness indicators; perform one or more loudness scaling tests during which sounds are delivered to a user of a hearing device; obtain results of the one or more loudness scaling tests via the first set of loudness indicators; and based on the results of the one or more loudness scaling tests, adapt the first set of loudness indicators to a second set of loudness indicators for use in at least one additional loudness scaling test.

In another aspect, a method is provided. The method comprises: performing a first set of intensity level tests during which stimulation signals are delivered to a user via a sensory prosthesis; during the first set of intensity level tests, displaying a first set of response options to the user; receiving, via the first set of response options, indications of a perceived loudness of the stimulation signals delivered to the user during the first set of intensity level tests; and adapting, based on the indications of a perceived loudness of the stimulation signals delivered to the user during the first set of intensity level tests, the first set of response options to a second set of response options for use during a second set of intensity level tests, where the first set of response options are associated with a first response precision level, and the second set of response options are associated with a second response precision level.

In another aspect a system is provided. The system comprises: a sensory prosthesis configured to deliver stimulation signals to a user during a first set of intensity scaling tests; a computing device comprising one or more processors configured to: during the first set of intensity scaling tests, display a first set of response options to the user via a display screen of the computing device; receive, via the first set of response options, indications of the user's perceived loudness of the stimulation signals delivered to the user during the first set of intensity scaling tests; and based on the indications of the user's perceived loudness of the stimulation signals delivered to the user during the first set of intensity scaling tests, adapt the first set of response options to a second set of response options for use during one or more second intensity scaling tests.

### BRIEF DESCRIPTION OF THE DRAWINGS

Embodiments of the present invention are described herein in conjunction with the accompanying drawings, in which:

FIGS. 1A and 1 are schematic diagrams illustrating an example sensory prosthesis fitting system that includes a sensory prosthesis that can benefit from the use of certain techniques presented herein;

FIG. 2A is a flowchart of an example method, in accordance with certain embodiments presented herein;

FIG. 2B is a flowchart of another example method, in accordance with certain embodiments presented herein;

FIGS. 3A and 3B illustrate first and second user interfaces, respectively, that provide different response options to a user, in accordance with certain embodiments presented herein;

FIGS. 4A, 4B, and 4C illustrate first, second, and third user interfaces, respectively, that provide different response options to a user, in accordance with certain embodiments presented herein;

FIG. 5 is a flowchart of an example method, in accordance with certain embodiments presented herein;

FIG. 6 is a functional block diagram of an implantable stimulator system that can benefit from the technologies described herein;

FIG. 7 illustrates an example cochlear implant system that can benefit from use of the technologies disclosed herein;

FIG. 8 illustrates a retinal prosthesis system that comprises an external device, a retinal prosthesis and a mobile computing device; and

FIG. 9 illustrates an example of a suitable computing system with which one or more of the disclosed examples can be implemented.

### DETAILED DESCRIPTION

Presented herein are techniques for administering loudness or intensity level scaling tests associated with sensory prostheses. In accordance with the techniques presented herein, a first set of loudness or intensity level scaling tests are performed during which stimulation signals are delivered to a user via a sensory prosthesis. During the first set of loudness or intensity level scaling tests, a first set of response options are provided to the user of the sensory prosthesis, and the first set of response options are used to receive indications of the user's perceived loudness of the delivered stimulation signals. The indications of a perceived loudness of the stimulation signals, received via the first set of response options, are used to adapt the first set of response options to a second set of response options for use during a second set of loudness or intensity level scaling tests. The first set of response options are associated with a first response precision level, while second set of response options are associated with a second response precision level that is different from the first response precision level.

As described elsewhere herein, it is to be appreciated that the techniques presented herein may be implemented with a number of different types of medical devices, including a variety of sensory prostheses/devices. For example, the techniques presented herein may be implemented with a number of different hearing/auditory devices/prostheses, such as hearing aids, middle ear auditory prostheses, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes or retinal prostheses), sensors, pacemakers, drug delivery systems, defibrillators, functional electrical stimulation devices, catheters, seizure devices (e.g., devices for monitoring and/or treating epileptic events), sleep apnea devices, electroporation devices, etc.

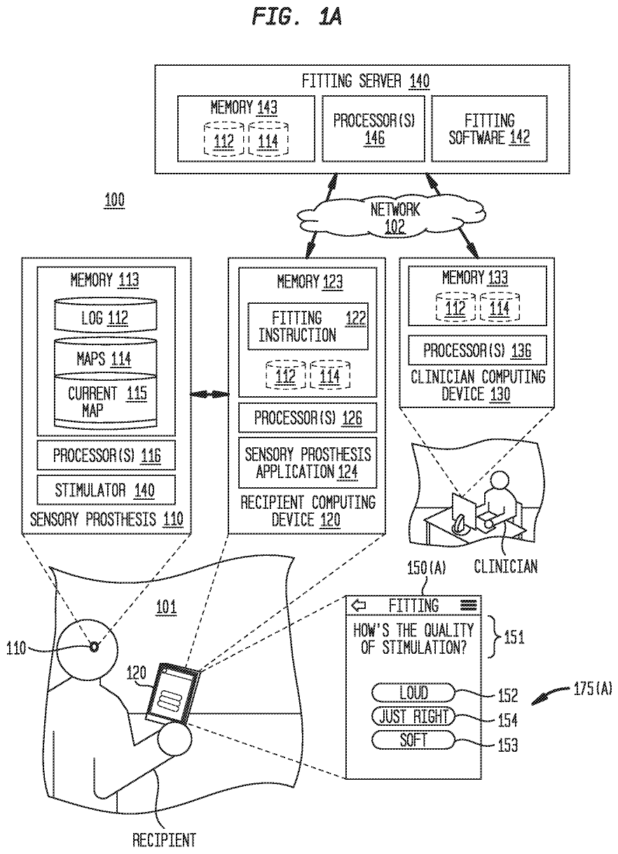

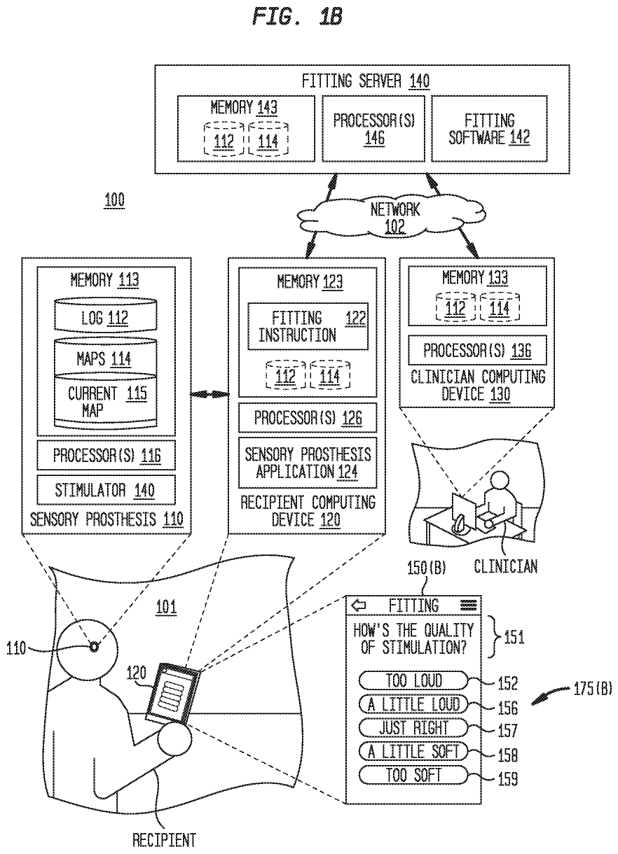

FIGS. 1A and 1B illustrate an example sensory prosthesis fitting system 100 that includes a sensory prosthesis 110 that can benefit from the use of technologies described herein. In this illustrative example, the sensory prosthesis fitting system 100 includes a user computing device 120, a clinician computing device 130, and a fitting server 140, which are connected with one another over a network 102. The network 102 may be, for example, a wired or wireless computer network (e.g., Local Area Network (LAN), Metropolitan Area Network (MAN), Wide Area Network (WAN), etc.), which facilitates the communication of data among the computing devices connected to the network.

As illustrated, the sensory prosthesis 110 and the user computing device 120 are operated by the user in an environment 101. The environment 101 defines the conditions in which the sensory prosthesis 110 and the user computing device 120 operate. In many examples herein, the environment 101 includes the auditory conditions in which the sensory prosthesis 110 functions. Such auditory conditions can include, for example, a loudness of noise in the environment (e.g., whether the environment 101 is loud or quiet). Other examples relate to the visual environment in which the sensory prosthesis 110 functions. Such visual conditions can include, for example, brightness or colors of the environment.

The sensory prosthesis 110 is a medical device/apparatus relating to a user's sensory system. For example, where the sensory prosthesis 110 is an auditory prosthesis, the sensory prosthesis 110 can be configured to provide stimulation to a user to cause auditory percepts based on a current map 115 and audio detected in the environment 101. Where the sensory prosthesis is a visual prosthesis, the sensory prosthesis 110 can be configured to provide stimulation to a user to cause visual percepts based on a current map 115 and light detected in the environment 101.

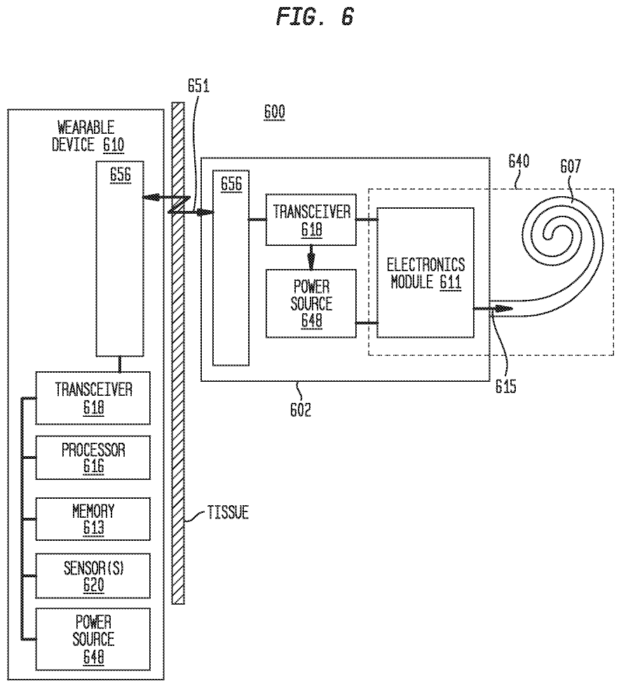

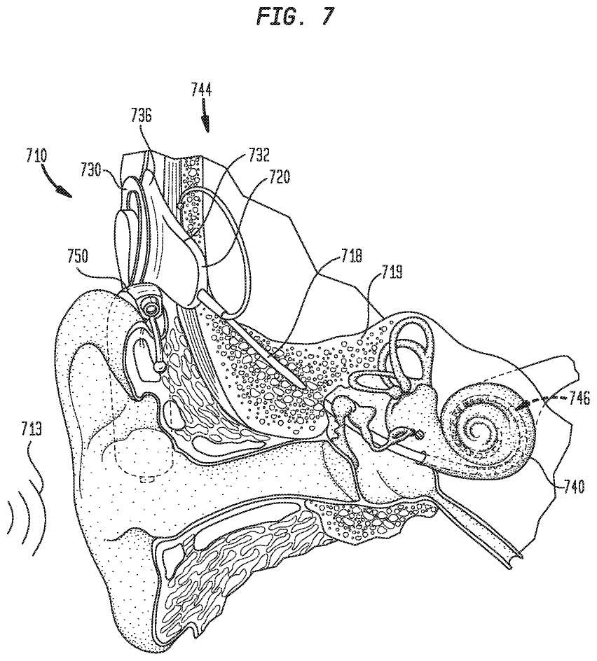

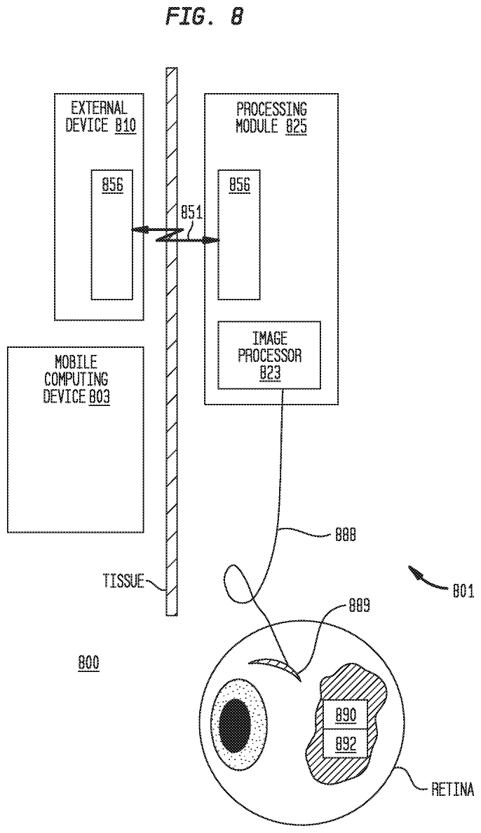

In an example, the sensory prosthesis 110 is an auditory prosthesis, such as a hearing aid, cochlear implant, bone conduction device (e.g., percutaneous bone conduction device, transcutaneous bone conduction device, active bone conduction device, passive bone conduction device, etc.), or a middle ear auditory prosthesis, among others. The sensory prosthesis 110 can take any of a variety of forms and examples are such forms are described in more detail in FIG. 6 (showing a stimulator device) and FIG. 7 (showing a cochlear implant). In an example, the sensory prosthesis 110 is a visual prosthesis, such as a retinal prosthesis (e.g., FIG. 8).

In the illustrated example, the sensory prosthesis 110 includes a memory 113, one or more processors 116, and a stimulator 140, among other components. In many examples, the sensory prosthesis 110 is a stimulator configured to cause the user to experience a sensory percept.

In the illustrated example, the memory 113 stores a log 112 and one or more maps 114. The log 112 is a set of one or more data structures that are records of data, activity, or events relevant to the sensory prosthesis 110. In an example, the log 112 includes data regarding multiple fitting sessions. The one or more data structures of the log can be implemented in any of a variety of ways.

The maps 114 are one or more settings for the sensory prosthesis 110. In an example, the one or more maps 114 describes an allocation of frequencies from a filter bank or other frequency analyzer to individual electrodes of the stimulator 140. In an example, the one or more maps 114 describe electrical maps from sound levels in one or more or all of the frequency bands to electrical stimulation levels. The one or more maps 114 can be performed on a one-to-one basis, with each filter output is allocated to a single electrode. The one or more maps 114 can be created based on parameters, such as threshold levels (T levels) and maximum comfort levels (C levels) for one or more or all stimulation channels of the sensory prosthesis 110. In an example, the one or more maps 114 are stored by programming the sensory prosthesis 110 or by any other process that sets the channels of the sensory prosthesis 110 to have the map 114. Example maps and related techniques are described in US 2008/0119910 and U.S. Pat. No. 9,757,562, which are hereby incorporated herein by reference in its entirety for any and all purposes.

The maps 114 can each be or include one or more parameters having values that affect how the sensory prosthesis 110 operates. For instance, the maps 114 can include a map 114 having minimum and maximum stimulation levels for frequency bands of stimulation channels. The map 114 is then used by the sensory prosthesis 110 to control an amount of stimulation to be provided. For instance, where the sensory prosthesis 110 is a cochlear implant, the map 114 affects which electrodes of the cochlear implant to stimulate and in what amount based on a received sound input. In some examples, the maps 114 include two or more predefined groupings of settings selectable by the user. One of the two or more predefined groupings of settings may be a default setting. In an example, the maps 114 can be ordered, such as based on relative loudness of the maps. For example, a first map 114 can have a lower loudness than an nth map 114, where n is the highest numbered map 114. In some examples, the differences between the maps 114 are simply intensity of stimulation. In other examples, there can be other differences between maps 114. In some implementations, the maps 114 can have different shapes compared to one another. For instance, the maps can be based on principle component analysis.

The maps 114 can also include sound processing settings that modify sound input before it is converted into a stimulation signal. Such settings can include, for example, particular audio equalizer settings can boost or cut the intensity of sound at various frequencies. In examples, the maps 114 can include a minimum threshold for which received sound input causes stimulation, a maximum threshold for preventing stimulation above a level which would cause discomfort, gain parameters, loudness parameters, and compression parameters. The maps 114 can include settings that affect a dynamic range of stimulation produced by the sensory prosthesis 110. As described above, many of the maps 114 affect the physical operation of the sensory prosthesis 110, such as how the sensory prosthesis 110 provides stimulation to the user in response to sound input received from the environment 101.

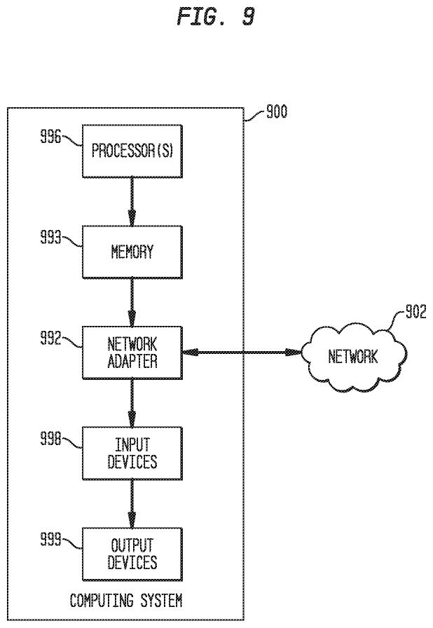

The one or more processors 116 include one or more hardware or software processors (e.g., microprocessors or central processing units). In many examples, the one or more processors 116 are configured to obtain and execute instructions from the memory 111. Additional details regarding the one or more processors 116 are described in relation to FIG. 9.

The stimulator 140 includes the stimulation generation and delivery components as well as additional support components of the sensory prosthesis 110. Examples include an electronics module and stimulation assembly, such as stimulation assemblies described in more detail below. As a specific example, the stimulator 140 is or includes an auditory stimulator. The auditory stimulator can be a component configured to provide stimulation to a user's auditory system to cause a hearing percept to be experienced by the user. Examples of components usable for auditory stimulation include components for generating air-conducted vibrations, components for generating bone-conducted vibration, components for generating electrical stimulation, other components, or combinations thereof.

The user computing device 120 is a computing device associated with the user of the sensory prosthesis 110. In many examples, the user computing device 120 is a mobile phone, tablet computer, laptop computer, smart watch, etc., but can take other forms. As illustrated, the user computing device 120 includes memory 123 and one or more processors 126.

As illustrated, the memory 123 includes fitting instructions 122. The fitting instructions 122 can be instructions executable by the one or more processors 126 of the user computing device 120 to implement one or more methods or operations described herein. In some examples, the fitting instructions 122 are a part of instructions executable to provide a sensory prosthesis application 124. In some examples, the memory 123 also stores the log 112 and one or more maps 114.

In examples, the user computing device 120 includes or implements the sensory prosthesis application 124 that operates on the user computing device 120 and cooperates with the sensory prosthesis 110. For instance, the sensory prosthesis application 124 can control the sensory prosthesis 110 (e.g., based on input received from the user) and obtain data from the sensory prosthesis 110. The user computing device 120 can connect to the sensory prosthesis 110 using, for example, a wireless radio frequency communication protocol (e.g., BLUETOOTH). The sensory prosthesis application 124 transmits or receives data from the sensory prosthesis 110 over such a connection. The sensory prosthesis application 124 can also stream audio to the sensory prosthesis 110, such as from a microphone of the user computing device 120 or an application running on the user computing device 120 (e.g., a video or audio application).

In some examples, the sensory prosthesis application 124 provides a fitting user interface, shown as user interface 150(A) in FIG. 1A and user interface 150(A). As described further below, the fitting user interface 150(A) is a first user interface configured to obtain fitting information from the user, while fitting user interface 150(A) is a second user interface configured to obtain fitting information from the user.

As illustrated, the fitting user interfaces 150(A) and 150(B) each include a query 151 to the user in the form of a text prompt. However, as shown, the user interfaces 150(A) and 150(B) include different numbers of response options, in the form of user interface elements (e.g., buttons), to obtain input/feedback from the user. More specifically, the user interface 150(A) includes a first set of response options 175(A) formed by three user interface elements that comprise includes a first user interface element 152 selectable to indicate that the stimulation is loud, a second user interface element 153 selectable to indicate that the stimulation is soft, and a third user interface element 154 selectable to indicate that the stimulation is just right. In contrast, the user interface 150(b) includes a second set of response options 175(B) formed by five user interface elements (e.g., buttons) that comprise a first user interface element 155 selectable to indicate that the stimulation is too loud, a second user interface element 156 selectable to indicate that the stimulation is a little loud, a third user interface element 157 selectable to indicate that the stimulation is just right, a fourth user interface element 158 selectable to indicate that the stimulation is a little soft, and a fifth user interface element 159 selectable to indicate that the stimulation is too soft. Other implementations of the user interfaces 150(A) and 150(B) are also usable.

As noted, the user interfaces 150(A) and 150(B) include different sets of response options (e.g., the first set of response options 175(A) in user interface 150(A) and the second set of response options 175(B) in user interface 150(B)). As described further below, the two sets of response options 175(A) and 175(B) provide different response “precision levels” for use during an adaptive loudness scaling process.

The clinician computing device 130 is a computing device used by a medical practitioner/professional that provides care or supervision for the user, such as a clinician, audiologist, etc. The clinician computing device 130 includes one or more software programs usable to monitor the sensory prosthesis 110, such as fitting progress thereof. The clinician computing device 130 can include memory 133 and one or more processors 136. In an example, the memory 133 stores instructions that, when executed by the one or more processors 136 causes the one or more processors 136 to obtain data regarding fitting of the sensory prosthesis 110 (e.g., via the server 140 or by a direct connection between the sensory prosthesis 110 or the user computing device 120 and the clinician computing device 130) and present such data to the clinician over a clinician user interface. In some examples, the data includes data stored in the log 112.

The fitting server 140 is a server computing device remote from the sensory prosthesis 110, user computing device 120, and the clinician computing device 130. The fitting server 140 is communicatively coupled to the user computing device 120 and the clinician computing device 130. In many examples, the fitting server 140 is indirectly communicatively coupled to the sensory prosthesis 110 through the user computing device 120 (e.g., via the sensory prosthesis application 124). In some examples, the fitting server 140 is directly communicatively coupled to the sensory prosthesis 110. The fitting server 140 includes memory 141, one or more processors 146, and fitting software 142. The fitting software 142 is software operable to perform one or more operations described herein, such as operations that fit the sensory prosthesis 110. The fitting software 142 can customize the sensory prosthesis 110 based on feedback from the user or the clinician.

In the examples of FIGS. 1A and 1, the memory 113, 123, 133, and 143 can each comprise one or more software-based or hardware-based computer-readable storage media operable to store information accessible by the one or more processors 116, 126, 136, and 146, respectively. The memory 113, 123, 133, and 143 can each comprise comprise/include RAM, ROM, EEPROM (Electronically-Erasable Programmable Read-Only Memory), flash memory, optical disc storage, magnetic storage, solid state storage, or any other memory media usable to store information for later access. In examples, the memory 113, 123, 133, and 143 can each encompass a modulated data signal (e.g., a signal that has one or more of its characteristics set or changed in such a manner as to encode information in the signal), such as a carrier wave or other transport mechanism and includes any information delivery media. By way of example, and not limitation, the memory 113, 123, 133, and 143 can each include wired media such as a wired network or direct-wired connection, and wireless media such as acoustic, RF, infrared and other wireless media or combinations thereof.

The components of the sensory prosthesis fitting system 100 can cooperate to perform one or more of the techniques presented herein. For example, in accordance embodiments presented herein, the sensory prosthesis 110 can be used to perform adaptive loudness scaling testing for determining (e.g., setting, adjusting, etc.) one more settings/attributes of the sensory prosthesis 110 (e.g., settings within maps 114). The adaptive loudness/intensity scaling tests can be used to, for example, determine the threshold levels (T levels) and maximum comfort levels (C levels) for one or more or all stimulation channels of the sensory prosthesis 110. The adaptive loudness/intensity scaling tests can be used to set/control, for example, loudness growth functions (Q level) for one or more or all stimulation channels of the sensory prosthesis 110, as the loudness growth function controls how the dynamic range of an input signal is translated to an electrical output. In certain embodiments, adaptive loudness/intensity scaling tests can be used to set or control, for example, target gain(s), maximum power output (MPO), gain curves/kneepoints, etc.

As used herein, a loudness or intensity scaling test is a type of test in which one or more stimulation signals are delivered to a user via a sensory prosthesis used by (e.g., worn by or implanted in) the user. During the loudness scaling test, the user is instructed (e.g., verbally, visually, etc.) to provide an indication of a perceived loudness of the one or more stimulation signals when delivered to the user via the sensory prosthesis.

In the specific context of a hearing device, a loudness or intensity scaling test is a type of audiological test in which stimulation signals representative of sounds (e.g., one or more tones) are delivered to at least one ear of the user via the hearing device used by (e.g., worn by or implanted in) the user. That is, the hearing device is used to evoke a hearing percept at one or more ears of the user. During the loudness scaling test, the user is instructed (e.g., verbally, visually, etc.) to provide an indication of a perceived loudness of the stimulations, when delivered to the user via the hearing device. During a loudness or intensity scaling test, the sounds can be generated by the hearing device, captured by the sound inputs (e.g., microphones of the hearing device), received from an external computing device (e.g., streamed via BLUETOOTH, via a connected cable, etc.), etc.

As described further below, “adaptive” loudness scaling testing references to a process in which a plurality of loudness scaling tests are administered to a user over time. During a first group of the loudness scaling tests, the user is presented with a first set (e.g., a minimized set) of possible responses (e.g., two or three possible responses) for use in the loudness scaling test/task. However, during a second group of the loudness scaling tests, the user is presented with a second set (e.g., an enlarged set) of possible responses (e.g., five or seven possible responses) for use in the loudness scaling test/task. Stated differently, the responses provided to the user are “adapted” between the first group of the loudness scaling tests and the second group of the loudness scaling tests to provide the user with a greater number of response options and, accordingly, provide more precision/granularity in the user's responses/feedback (e.g., adapt how responses are elicited from the user as the hearing precision/discrimination testing evolves).

The initial use of a minimized set of responses is based on an understanding that most new sensory prosthesis users have trouble perceiving loudness differences with sufficient granularity to provide specific/precise feedback. That is, when use of a sensory prosthesis is new for a user, the user may only be able to detect course/gross loudness differences.

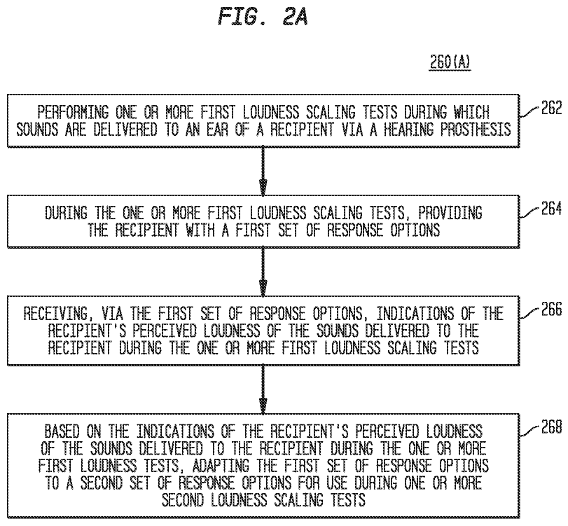

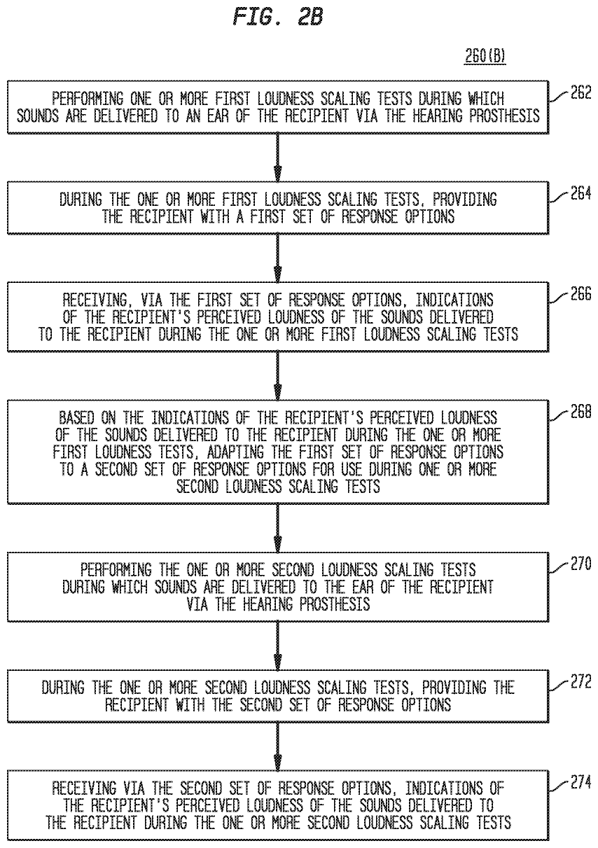

However, it has been determined that, as a user becomes more comfortable with her sensory prosthesis over time (e.g., through continued use, training, etc.), the user can usually perceive increasingly subtle loudness differences. This understanding is leveraged in the techniques presented herein by increasing the granularity/precision in the response options over time to corresponding obtain increasingly precision/granularity in the user's responses/feedback. Example such techniques are described below with reference to FIGS. 2A and 2B.

More specifically, FIG. 2A is a flowchart of an example method 260(A), in accordance with certain embodiments presented herein. For ease of description, method 260(A) is described with reference to with reference to the arrangement of FIGS. 1A and 1, where the sensory prosthesis 110 is a hearing device and, as such, the fitting system 100 is a hearing device fitting system.

Method 260(A) begins at 262 where the hearing device fitting system 100 performs a first set of one or more first loudness scaling tests. At 266, the hearing device fitting system 100 receives, via the first set of response options (e.g., user interface 150(A), indications of the user's perceived loudness of the sounds delivered to the user during the one or more first loudness scaling tests. At 268, based on the indications of the user's perceived loudness of the sounds delivered to the user during the one or more first loudness tests, the first set of response options are adapted to a second set of response options for use during one or more second loudness scaling tests. In one example, the second set of response options are illustrated by user interface 150(B) of FIG. 1B.

FIG. 2B illustrates another example method, referred to as method 206(B), that is an extension of method FIG. 2A. More specifically, method 260(B) includes the operations of 262, 264, 266, and 268, but also includes operations at 270, 272, and 274. In this example, at 270, the hearing device fitting system 100 performs the one or more second loudness scaling tests. As shown, at 272, during the one or more second loudness scaling tests, the user is provided with the second set of response options (e.g., user interface 150(B) of FIG. 1). At 274, the hearing device fitting system 100 receives, via the second set of response options, indications of the user's perceived loudness of the sounds delivered to the user during the one or more second loudness scaling tests.

In summary, FIGS. 2A and 2B generally illustrate that, in accordance with certain embodiments presented herein, a user can be initially be presented with a first (e.g., minimized/reduced) set responses (e.g., two or three possible responses) for use during a first set of loudness scaling tests/tasks. As noted above, the initial use of a minimized set of responses is based on an understanding that most new hearing device users have trouble perceiving loudness differences with sufficient granularity to provide specific/precise feedback. That is, when use of a hearing device is new for a user, the user may only be able to detect course/gross loudness differences, such as only able to determine if a sound is soft, loud, or neither soft nor loud (e.g., just right or acceptable).

However, also as noted above, it has been determined that, as a user becomes more comfortable with her hearing device over time (e.g., through continued use, training, etc.), the user can usually perceive increasingly subtle loudness differences. This understanding is leveraged in the techniques presented herein by increasing, over time, the granularity/precision level in the set of response options presented to the user to, accordingly, obtain increasingly precision/granularity in the user's responses/feedback. As used herein, increasing the “granularity level” or “precision level” in the set of response options refers to changes that allow the user to provide relatively more precise feedback. The precision level in the set of response options can be increased, for example, by increasing the number of response options that are presented to the user (e.g., adapting a first set of three response options to a second set of five response options, increasing a second set of five response options to a third set of seven response options, etc.)

The precision level of the response options is automatically adapted, by an automated adaption module, based on the user's responses to the loudness/intensity scaling tests. For example, in certain embodiments, a certain number or percentage of responses that correlate with expected responses may indicate that the user's perception has progressed to a point at which the precision level of the response options should be adapted. Conversely, a certain number or percentage of responses that fail correlate with expected responses may indicate that the user's perception is insufficient to increase precision or may indicate that the precision level of the response should be decreased (e.g., revert back from a second set of five response options to a first set of three response options, etc.).

In certain examples, the precision level of the response options is automatically adapted using a machine learning model or other type of Artificial Intelligence (AI) system, such as an Artificial Neural Network (ANN). That is, the automated adaption module comprises an AI system that is configured to analyze the user's loudness responses, along with individualized user data, to adapt the precision level of the responses. In certain such examples, some statistics in the amount of “correct” responses, a weighed comparison of responses to responses from normative data, etc. would enable the adaption to the next or former level of granularity. In certain embodiments, additional types of testing, such as phoneme discrimination tests, could be used to control the adaption. For example, phoneme discrimination tests could be used to identify problem frequencies and the testing could be adapted to focus on those specific frequencies before advancing to a next level.

The individualized user data can comprise, for example, personal attributes/data associated with a specific user, such as the user's age, medical condition(s), language, location(s), current device settings, typical sound environments, preferences, etc. of the specific user. The individualized user data can also include other factors, such as the specific user's psychoacoustic characteristics, family genetic history, personal medical background, etc. In the example of FIGS. 1A and 1, the individualized user data may be part of the user's log 112 and the automated adaption module may be part of the fitting software 142. Although described as being part of fitting software 142 in FIGS. 1A and 1B, the an automated adaption module could also or alternatively be implemented at the user computing device 120 and/or the clinician computing device 130.

The AI system (e.g., automated adaption module) is trained to perform the adaption based on correlated normative data. In general, the correlated normative data is historical data obtained from a large population of different sensory device users, which has been analyzed and associated together in a meaningful way based on one or more factors or metrics. The correlated normative data may comprise, for example, different types of audiological data that is correlated based on different types of individualized user data (e.g., audiograms correlated by hearing loss type and age). The correlated normative data can be embodied as a pre-built database or that is updated periodically/dynamically (e.g., in real-time) in response to user fittings, testing, etc., and be used to periodically/dynamically re-train or update the AI system. The correlated normative data may, for example, be part of the fitting software 142.

As noted, in accordance with the embodiments presented herein, a user is provided, overtime, a variable number of response options that start with a first/initial (minimal) response set (e.g., [SILENT/TSPL/CSPL], [CAN'T HEAR SOFT/LOUD], etc.) that is subsequently adapted or evolved to one or more other advanced response sets that provide greater levels of precision, relative to the initial response set and/or other response sets based on response consistency. It is to be appreciated that the initial response set and the one or more advanced response sets can have a number of different forms/arrangements.

As noted, user interface 150(A) illustrates one example of a first set of response options, while user interface 150(B) illustrates an example of a second set of response options where the second set of response options provide a greater level of precision in the perceived loudness than a level of precision in the perceived loudness provided by the first set of response options (e.g., the first set of response options include a first number of possible responses and the second set of response options include a second number of possible responses, wherein the second number of possible responses is larger than the first number of possible responses).

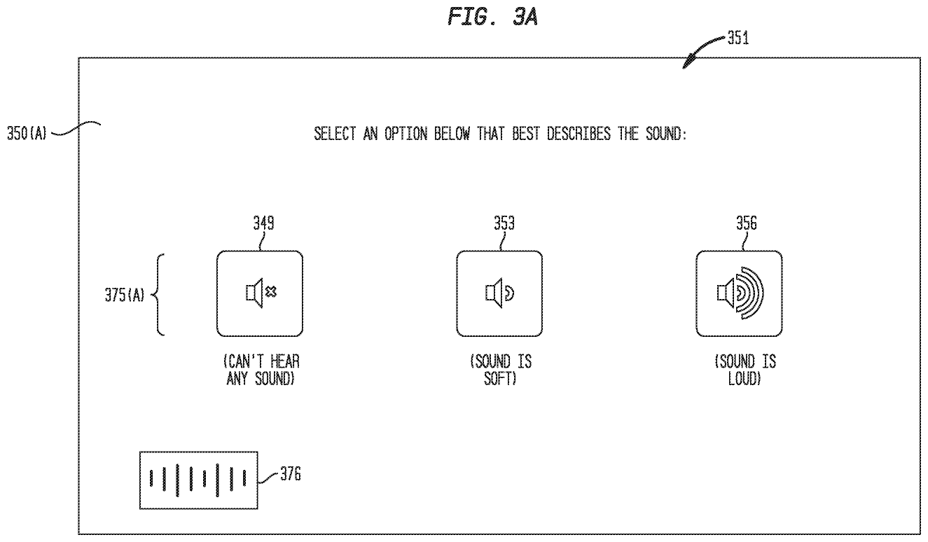

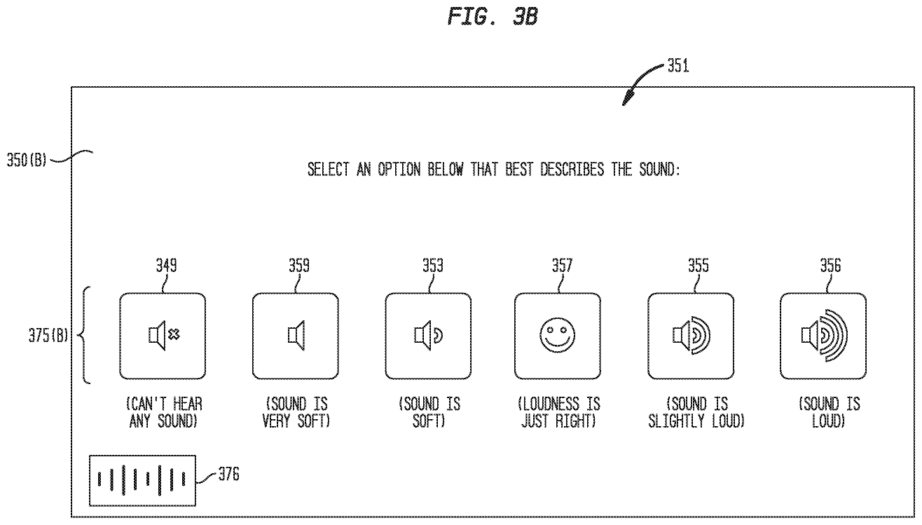

FIG. 3A illustrates another example of a user interface 350(A) that provides a first set of response options 375(A) to a user, while FIG. 3B illustrates another example of a user interface 350(B) that provides a second set of response options 375(B) where the second set of response options provide a greater level of precision in the perceived loudness than a level of precision in the perceived loudness provided by the first set of response options (e.g., the first set of response options include a first number of possible responses and the second set of response options include a second number of possible responses).

More specifically, in user interface 350(A), the first set of response options 375(A) are formed by three user interface elements (e.g., selectable icons) that comprise a first user interface element 349 selectable to indicate that a sound is inaudible (e.g., “I can't hear any sound”), a second user interface element 353 selectable to indicate that the sound is soft, and a third user interface element 356 selectable to indicate that the sound is loud.

In FIG. 3A, the user interface elements 349, 353, and 356 are accompanied by explanatory text for the benefit of the user. However, in other embodiments, the explanatory text can be omitted. As shown, the user interface 350(A) also includes a text prompt 351 (e.g., instructions or a query) for the user in to make a selection of one of the response options 375(A) and an icon 376 indicating when a response is requested (e.g., following delivery of a sound). The use of the icon 376 is merely illustrative and that other icons or other techniques may be used to indicate when a when a response is requested (e.g., flashing text prompt 351, change in color at the display, etc.).

In user interface 350(B), the second set of response options 375(B) are formed by six user interface elements (e.g., selectable icons) that, similar to FIG. 3A, includes the first user interface element 349 selectable to indicate that a sound is inaudible (e.g., “I can't hear any sound”), the second user interface element 353 selectable to indicate that the sound is soft, and the third user interface element 356 selectable to indicate that the sound is loud. However, the second set of response options 375(B) also includes a fourth user interface element 359 to indicate that the sound is very soft (e.g., a loudness between “soft” and “inaudible), a fifth user interface element 357 to indicate that the loudness of the sound is just right (e.g., comfortable), and a sixth user interface element 355 to indicate that the sound is slightly loud (e.g., a loudness between “just right” and “loud,”).

Again, in the example of FIG. 3B, the user interface elements 349, 359, 353, 357, 355, and 356 are accompanied by explanatory text for the benefit of the user. However, in other embodiments, the explanatory text can be omitted. As shown, the user interface 350(B) also includes the text prompt 351 and the icon 376.

As noted, the user interfaces 350(A) and 350(B) include different numbers of response options, in the form of user interface elements (e.g., buttons), to obtain input/feedback from the user. The second set of response 375(B) provides a greater level of precision in responses from the user than the first set of responses 375(A). In particular, and as noted above, the second set of response 375(B) include a greater number of response options than the first set of response options 375(A). However, as noted elsewhere herein, the use of different numbers of response options is merely one illustrative technique for providing different response precision levels during an adaptive loudness scaling process.

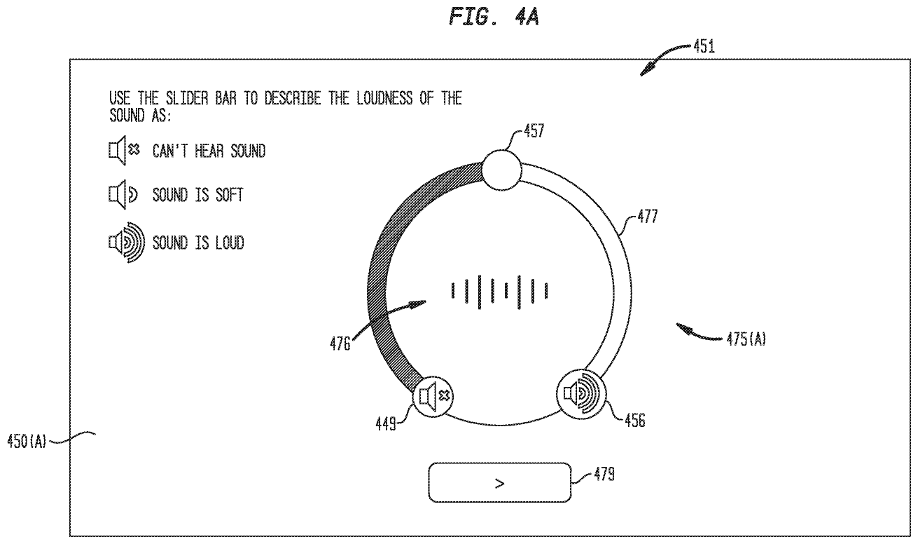

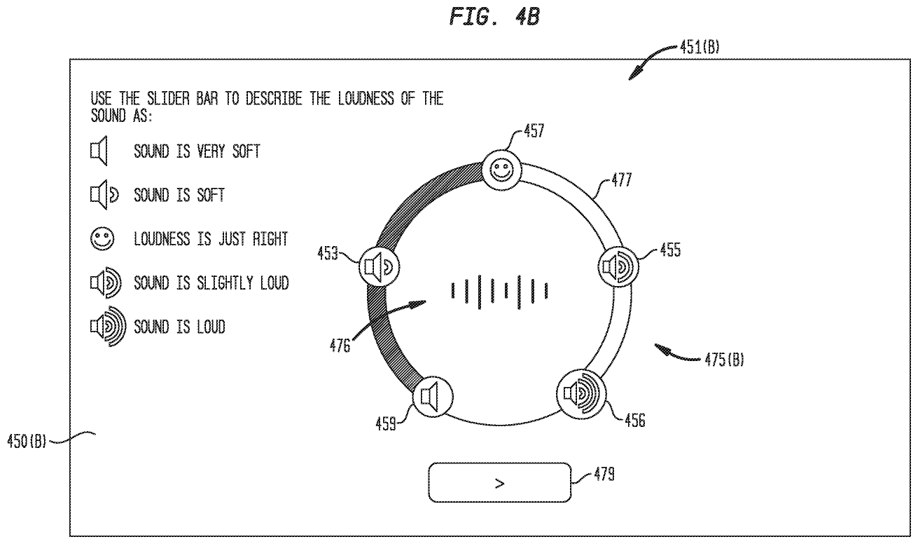

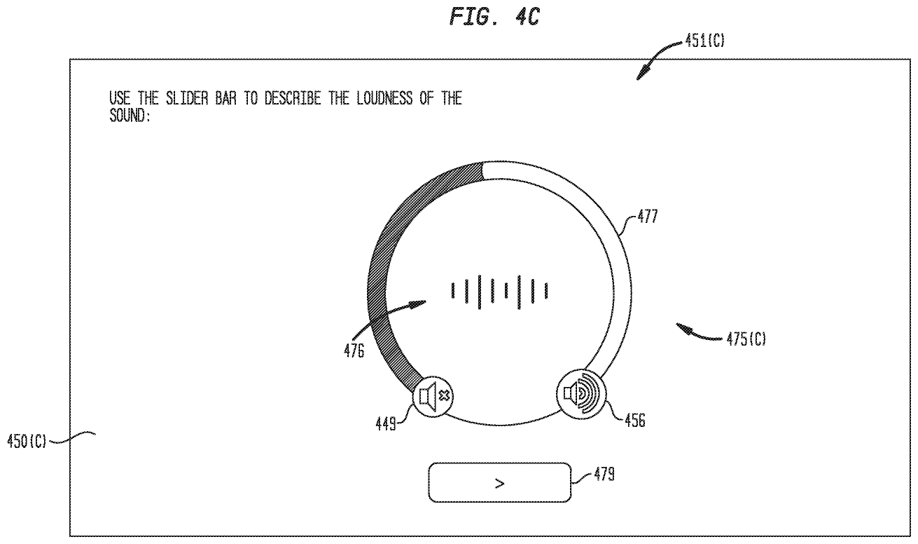

For example, FIG. 4A illustrates another example of a user interface 450(A) that provides a first set of response options 475(A) to a user, FIG. 4B illustrates another example of a user interface 450(B) that provides a second set of response options 475(B), and FIG. 4C illustrates an example of a user interface 450(B) that provides a third set of response options 475(C). As described below, the third set of response options 475(C) provides a greater level of precision in the perceived loudness than a level of precision in the perceived loudness provided by the second set of response options 475(B). Similarly, the second set of response options 475(B) provides a greater level of precision in the perceived loudness than a level of precision in the perceived loudness provided by the first set of response options 475(A).

More specifically, in user interface 450(A), the first set of response options 475(A) are formed by a single integrated interface element in form of a fillable slider bar 477 displayed at a touchscreen. In this example, the user can use her finger, stylus, etc. to “fill” the slider bar 477 and thereby indicate a perceived loudness of a delivered sound. However, in the example of FIG. 4A, the slider bar 477 only includes three discrete positions that can be selected by the user, namely that the slider is empty (represented at point 449), half full (represented at point 453), or full (represented by point 456). Stated differently, in the example of FIG. 4A, the user can only select from points 449, 453, and 456. Point 449 is selectable to indicate that a sound is inaudible (e.g., “I can't hear any sound”), the second point 453 selectable to indicate that the sound is soft, and the third point 456 is selectable to indicate that the sound is loud. In the specific example of FIG. 4A, the user actuated the slider bar 477 to point 453, thereby indicating that the delivered sound is perceived to be “soft.”

As shown, the user interface 450(A) also includes a text prompt 451(A) (e.g., instructions or a query) for the user in to actuate the slider bar 477 and select one of the points 449, 453, or 456, along with a description of each point. In the specific example of FIG. 4A, the user interface 450(A) also includes an icon 476 indicating when a response is requested (e.g., following delivery of a sound). The use of the icon 476 is merely illustrative and that other icons or other techniques may be used to indicate when a when a response is requested (e.g., flashing text prompt 451(A), change in color at the display, etc.). The user interface 450(A) also comprises a play icon 479 that can be used to trigger the delivery of a sound to the user via the hearing device in, for example, a self-fitting/self-testing arrangement.

Similar to the example of FIG. 4A, the second set of response options 475(B) of user interface 450(B) in FIG. 4B are formed by fillable slider bar 477 displayed at the touchscreen. Again, the user can use her finger, stylus, etc. to “fill” the slider bar 477 and thereby indicate a perceived loudness of a delivered sound. However, in the example of FIG. 4B, the slider bar 477 includes five discrete positions that can be selected by the user, namely that the slider is empty (represented at point 459), one-fourth full (represented at point 453), half full (represented at 457) three-fourths full (represented by point 455), and full (represented at point 456). Stated differently, in the example of FIG. 4A, the user can only select from points 459, 453, 457, 455, and 456. The first point 459 is selectable to indicate that a sound is very soft, the second point 453 selectable to indicate that the sound is soft, the third point 457 is selectable to indicate that the loudness of the sound is just right, the fourth point 455 is selectable to indicate that the sound is slightly loud, and the fifth point 456 is selectable to indicate that the sound is loud. In the specific example of FIG. 4A, the user actuated the slider bar 477 to point 457, thereby indicating that the loudness of the delivered sound is “just right.”

As shown, the user interface 450(B) also includes a text prompt 451(B) for the user to actuate the slider bar 477 and select one of the points 459, 453, 457, 455, or 456. In the specific example of FIG. 4B, the user interface 450(B) also includes the icon 476 indicating when a response is requested (e.g., following delivery of a sound) and the play icon 479 that can be used to trigger the delivery of a sound to the user via the hearing device.

As shown, the user interface 450(B) provides a greater precision level in user response options 475(B), relative to the response options 475(A) provided in user interface 450(A) (e.g., five possible response options versus three possible response options). However, it is also noted that the response options 475(B) shown in FIG. 4B are not the same as the response options 475(A) shown in FIG. 4B. For example, whereas the response options 475(A) of FIG. 4A begin at 449 (inaudible), the response options 475(B) of FIG. 4B begin at 459 (very soft). This difference reflects that, as a user becomes more comfortable with her hearing device, there may no longer be a need to test/determine whether the user can hear a sound or not, whereas such testing may be important for new users to, for example, ensure the hearing device is working properly, to determine gross threshold levels, etc.

FIG. 4C illustrates another example in which, similar to the example of FIG. 4A, the third set of response options 475(C) of user interface 450(C) are formed by fillable slider bar 477 displayed at the touchscreen. Again, the user can use her finger, stylus, etc. to “fill” the slider bar 477 and thereby indicate a perceived loudness of a delivered sound. However, in the example of FIG. 4C, the slider bar 477 includes only a beginning position/point 449 and an ending position/point 456, but allows the user to stop the slider any position between those two points.

More specifically, in the example of FIG. 4C, the user can leave the slider bar 477 empty (represented at point 449) to indicates that the sound is inaudible, or fill the slider bar 477 (represented at point 456) to indicate that the sound is loud. However, the user can also actuate the slider bar 477 so that it is filled to any point between 449 and 456, to rank or rate the loudness of the sound. As such, in the example of FIG. 4C, the user is provided with a large number of response options 475(C) (e.g., point 449, point 456, or any point there between).

In the example of FIG. 4C, the slider bar 477 is shown filled to roughly 50%. In operation, a system would be configured to detect how much the user “filled” the slider bar 477, and then determine the perceived loudness therefrom.

As shown, the user interface 450(C) also includes a text prompt 451(C) for the user in to actuate the slider bar 477 to describe a loudness of the sound. In the specific example of FIG. 4C, the user interface 450(B) also includes the icon 476 indicating when a response is requested (e.g., following delivery of a sound) and the play icon 479 that can be used to trigger the delivery of a sound to the user via the hearing device.

The user interface 450(C) of FIG. 4C illustrates an example of an advanced interface for experienced hearing device users who have the ability to differentiate between subtle loudness differences. Whereas the response options 475(C) could be overwhelming for new users, these response options 475(C) could enable an experienced user to perform self-fitting to precisely refine her device settings.

As shown, the user interface 450(B) provides a greater precision level in user response options 475(C), relative to both the response options 457(A) provided in user interface 450(A) and the response options 475(B) provided in user interface 450(B). The user interface 450(C) of FIG. 4C illustrates an example of an advanced interface for experienced hearing device users who have the ability to differentiate between subtle loudness differences. Such an interface may enable an experienced user to perform self-fitting to precisely refine her device settings.

As noted above, the adaptive loudness scaling techniques described herein generally present a first set of response options in user interface for use during a first set of loudness scaling tests, but present a second set of response options in a user interface for use during a second set of loudness scaling tests. Also as noted, the first and second sets of response options provide different response precision levels. In certain embodiments, the second set of loudness scaling tests may be the same as, or substantially similar to, the first set of loudness scaling tests. In other embodiments, the second set of loudness scaling tests may be different from the first set of loudness scaling tests. For example, the “granularity” or “precision” level of the loudness scaling tests could be increased in the loudness domain or the frequency domain.

In general, increasing the precision of the loudness scaling tests in the frequency domain refers to narrowing, over time, the frequency bands associated with the sound signals presented to the user such that the loudness scaling is associated with increasingly fewer stimulation channels. More specifically, in certain examples, the first set of loudness scaling tests could be broadband loudness scaling tests performed using broadband signals that would be configured to result setting threshold and/or comfort levels across the entire frequency range of the hearing device. That is, the user is presented with a broadband sound signal and tasked with indicating the loudness of the broadband signals. The user's responses could then be used to set (e.g., raise/lower) threshold or comfort levels across the entire frequency spectrum of the hearing device. The user's responses could also be used to control the loudness growth function (Q level) across the entire frequency spectrum of the hearing device. The user's responses could also be used to control for example, target gain(s), maximum power output (MPO), gain curves/kneepoints, etc. across the entire frequency spectrum of the hearing device.

However, in these embodiments, the second set of loudness scaling tests could be narrowband loudness scaling tests performed using discrete (narrower) frequency bands. That is, the user is presented with a number of different sound signals, where each sound is associated with only a discrete frequency band and tasked with indicating the loudness of each of the sound signals. The user's responses could then be used to set (e.g., raise/lower) threshold or comfort levels for only the frequency band with which a given sound signal is associated. The user's responses could also be used to control the loudness growth function associated with the frequency band with which a given sound signal is associated. The user's responses could also be used to control for example, target gain(s), maximum power output (MPO), gain curves/kneepoints, etc. associated with the frequency band with which a given sound signal is associated.

The frequency adaption of the loudness scaling tests (e.g., switch from the use of broadband loudness scaling tests to narrowband loudness scaling tests) can occur over time in a progressive and/or adaptive manner and is based on the user's responses. That is, the frequency adaption is tailored to the specific user and may be controlled, for example, by the automated adaption module described elsewhere herein. Moreover, it the frequency adaption can occur in a stepwise manner that progressions from use of the broadband signal to increasingly narrower frequency signals over time (e.g., start with broadband signals and eventually adapt to use of ⅓ octave bands [250/500/1000/2000/4000/6000]).

As noted, adaption of the loudness scaling tests in the frequency domain (e.g., progressively adapting from the use of broadband loudness scaling tests to narrowband loudness scaling test) is merely one illustrative technique for adapting the precision level of the loudness scaling tests. In another example, the loudness scaling tests could be adapted in the loudness domain.

In general, increasing the precision of the loudness scaling tests in the loudness domain refers to the addition of more granularity in the presented signals (e.g., steps of 20 dB between 20 and 80 dB with the first set loudness scaling tests, but steps of 10 dB between 20 and 80 dB in the second set of loudness scaling tests). The increase in the precision of the loudness scaling tests in the loudness domain can occur over time in a progressive and/or adaptive manner and is based on the user's responses. For example, the loudness adaption may be controlled by the automated adaption module described elsewhere herein. Moreover, it the loudness adaption can occur in a stepwise manner that progressions from use of the large loudness differences to increasingly smaller loudness differences over time.

Additionally, the precision of the loudness scaling tests could be increased through the addition of binaural loudness scaling tests. For example, for a binaural user, the loudness scaling could be initially performed separately/independently at the hearing prostheses located at each of the left and right ears. Once the left and right have reached independent stable levels, the loudness scaling process could be performed for both hearing devices at the same time (e.g., binaural loudness scaling tests). Whereas performing such binaural loudness scaling tests early on in the fitting journey would be difficult for new users, such binaural loudness scaling tests could be valuable for experienced users in, for example, determining ILD settings that improve localization of sounds.

The above examples have generally between described in terms of increasing the precision level of the response options and/or increasing the precision level of the loudness scaling tests. However, it is to be appreciated that, in certain embodiments presented herein, the precision level of the response options and/or increasing the precision level of the loudness scaling tests could also be decreased (devolved). For example, if a user's responses are not consistent or above some expected level, the system could decrease the granularity of responses and/or stimuli and continue to test at the lowered level. As such, “adaption” of the precision level of the response options and/or adaption the precision level of the loudness scaling tests may refer to increasing or decreasing the precision levels of the response options and/or scaling tests.

As noted above, presented herein are an adaptive loudness scaling processes, with a testing and management system, for use with sensory prostheses, such as cochlear implants or other hearing device. For hearing prostheses, in particular, determining the correct loudness (e.g., threshold and comfort levels, loudness growth functions, etc.) for the different stimulation channels is fundamental to the fitting process. In accordance with the techniques presented herein, a user is able to, using a testing device, initially responds to broadband signals by selecting from a limited number of loudness classes. As the user develops and becomes more sensitive to fine changes in the stimulus and/or becomes more confident in using the new device and technology, adaptations are made so that the user is ask to respond to tests that are more complex and/or nuanced, while being presented with more response options. For example, there may be an increase in the number of loudness category options available to be selected by the user. As noted, another example of adaption is to change the test signals from broadband to narrowband, to thus enable further nuanced measuring and tracking of the user's electric hearing development. Another example is to permit the user to view and select new testing features as they become more confident in using the technology. The degree and kind of adaptation can be driven in accordance with the user's measured progress and/or may take other factors into account such as time. As a result, the techniques presented provide a more accurate and relevant way to measure and rehabilitate electric hearing development, which is characteristically different especially for loudness as compared to natural or acoustic hearing.

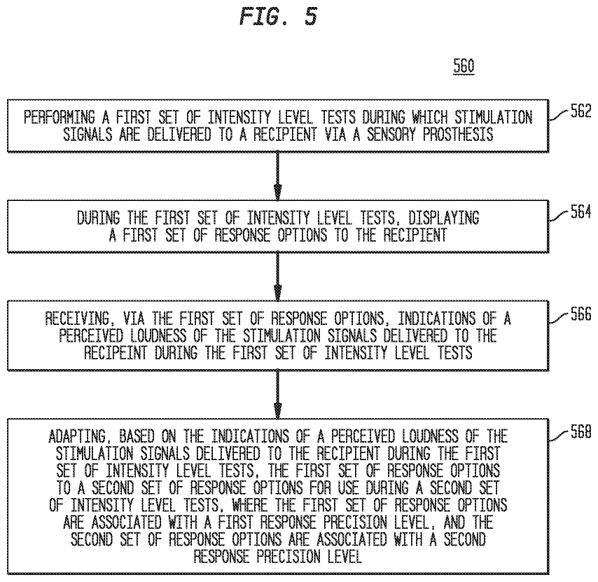

FIG. 5 is a flowchart of an example method 560, in accordance with certain embodiments presented herein. Method 560 begins at 562 where a first set of intensity level tests are performed during which stimulation signals are delivered to a user via a sensory prosthesis. At 564, during the first set of intensity level tests, one or more processors display a first set of response options to the user. At 566, the one or more processors receive, via the first set of response options, indications of a perceived loudness of the stimulation signals delivered to the user during the first set of intensity level tests. At 568, the one or more processors adapt, based on the indications of a perceived loudness of the stimulation signals delivered to the user during the first set of intensity level tests, the first set of response options to a second set of response options for use during a second set of intensity level tests. The first set of response options are associated with a first response precision level, and the second set of response options are associated with a second response precision level that is different from the first response precision level. In the example of FIG. 5, the one or more processors can be implemented a computing device or distributed across a plurality of computing devices (e.g., a mobile phone and server, or other combinations).

As noted above, the techniques disclosed herein can be applied in any of a variety of circumstances and with a variety of different devices. For example, the sensory prosthesis 110 can take the form of a variety of different consumer devices or medical devices. Example consumer devices include headphones, earbuds, personal sound amplification products, wireless earbuds, or other consumer devices. Example medical devices include auditory prostheses and visual prostheses. Example auditory prostheses include one or more prostheses selected from the group consisting of: a cochlear implant, an electroacoustic device, a percutaneous bone conduction device, a passive transcutaneous bone conduction device, an active transcutaneous bone conduction device, a middle ear device, a totally-implantable auditory device, a mostly-implantable auditory device, an auditory brainstem implant device, a hearing aid, and a tooth-anchored hearing device. Example visual prostheses include bionic eyes.

Specific example devices that can benefit from technology disclosed herein are described in more detail in FIGS. 6-8, below. For example, the techniques described herein can be used to with different medical devices, such as an implantable stimulation system as described in FIG. 6, a cochlear implant as described in FIG. 7, or a retinal prosthesis as described in FIG. 8.

More specifically, FIG. 6 is a functional block diagram of an implantable stimulator system 600 that can benefit from the technologies described herein. In an example, the sensory prosthesis 110 corresponds to the implantable stimulator system 600. The implantable stimulator system 600 includes a wearable device 610 acting as an external processor device and an implantable device 650 acting as an implanted stimulator device. In examples, the implantable device 650 is an implantable stimulator device configured to be implanted beneath a user's tissue (e.g., skin). In examples, the implantable device 650 includes a biocompatible implantable housing 602. Here, the wearable device 610 is configured to transcutaneously couple with the implantable device 650 via a wireless connection to provide additional functionality to the implantable device 650.

In the illustrated example, the wearable device 610 includes one or more sensors 620, a memory 613, at least one processor 616, a transceiver 618, and a power source 648. The one or more sensors 620 can be units configured to produce data based on sensed activities. In an example where the stimulation system 600 is an auditory prosthesis system, the one or more sensors 620 include sound input sensors, such as a microphone. Where the stimulation system 600 is a visual prosthesis system, the one or more sensors 620 can include one or more cameras or other visual sensors. The processor 116 can be a component (e.g., a central processing unit) configured to control stimulation provided by the implantable device 650. The stimulation can be controlled based on data from the sensor 620, a stimulation schedule, or other data. Where the stimulation system 600 is an auditory prosthesis, the processor 116 can be configured to convert sound signals received from the sensor(s) 130 (e.g., acting as a sound input unit) into signals 651. The transceiver 618 is configured to send the signals 651 in the form of power signals, data signals, combinations thereof (e.g., by interleaving the signals), or other signals. The transceiver 618 can also be configured to receive power or data. Stimulation signals can be generated by the processor 116 and transmitted, using the transceiver 618, to the implantable device 650 for use in providing stimulation.

In the illustrated example, the implantable device 650 includes a transceiver 618, a power source 648, a coil 656, and a stimulator 640 that includes an electronics module 611 and a stimulator assembly 607. The implantable device 650 further includes a hermetically sealed, biocompatible housing enclosing one or more of the components.

The electronics module 611 can include one or more other components to provide sensory prosthesis functionality. In many examples, the electronics module 611 includes one or more components for receiving a signal (e.g., from one or more of the sensors 620) and converting the signal into the stimulation signal 615. The electronics module 611 can further be or include a stimulator unit. The electronics module 611 can generate or control delivery of the stimulation signals 615 to the stimulator assembly 607. In examples, the electronics module 611 includes one or more processors (e.g., central processing units or microcontrollers) coupled to memory components (e.g., flash memory) storing instructions that when executed cause performance of an operation. In examples, the electronics module 611 generates and monitors parameters associated with generating and delivering the stimulus (e.g., output voltage, output current, or line impedance). In examples, the electronics module 611 generates a telemetry signal (e.g., a data signal) that includes telemetry data. The electronics module 611 can send the telemetry signal to the wearable device 610 or store the telemetry signal in memory for later use or retrieval.

The stimulator assembly 607 can be a component configured to provide stimulation to target tissue. In the illustrated example, the stimulator assembly 607 is an electrode assembly that includes an array of electrode contacts disposed on a lead. The lead can be disposed proximate tissue to be stimulated. Where the system 600 is a cochlear implant system, the stimulator assembly 607 is configured to be inserted into the user's cochlea. The stimulator assembly 607 can be configured to deliver stimulation signals 615 (e.g., electrical stimulation signals) generated by the electronics module 611 to the cochlea to cause the user to experience a hearing percept. In other examples, the stimulator assembly 607 is a vibratory actuator disposed inside or outside of a housing of the implantable device 650 and configured to generate vibrations. The vibratory actuator receives the stimulation signals 615 and, based thereon, generates a mechanical output force in the form of vibrations. The actuator can deliver the vibrations to the skull of the user in a manner that produces motion or vibration of the user's skull, thereby causing a hearing percept by activating the hair cells in the user's cochlea via cochlea fluid motion.

The transceivers 618 can be components configured to transcutaneously receive and/or transmit a signal 651 (e.g., a power signal and/or a data signal). The transceiver 618 can be a collection of one or more components that form part of a transcutaneous energy or data transfer system to transfer the signal 651 between the wearable device 610 and the implantable device 650. Various types of signal transfer, such as electromagnetic, capacitive, and inductive transfer, can be used to usably receive or transmit the signal 651. The transceiver 618 can include or be electrically connected to the coil 656.

The coils 656 can be components configured to receive or transmit a signal 651, typically via an inductive arrangement formed by multiple turns of wire. In examples, in addition to or instead of a coil, other arrangements are used, such as an antenna or capacitive plates. The magnets can be used to align respective coils 656 of the wearable device 610 and the implantable device 650. For example, the coil 656 of the implantable device 650 is disposed in relation to (e.g., in a coaxial relationship) with an implantable magnet set to facilitate orienting the coil 656 in relation to the coil 656 of the wearable device 610 via the force of a magnetic connection. The coil 656 of the wearable device 610 can be disposed in relation to (e.g., in a coaxial relationship) with a magnet set.

The power source 648 can be one or more components configured to provide operational power to other components. The power source 648 can be or include one or more rechargeable batteries. Power for the batteries can be received from a source and stored in the battery. The power can then be distributed to the other components of the implantable device 650 as needed for operation.

FIG. 7 illustrates an example cochlear implant system 710 that can benefit from use of the technologies disclosed herein. For example, the cochlear implant system 710 can be used to implement the sensory prosthesis 110. The cochlear implant system 710 includes an implantable component 744 typically having an internal receiver/transceiver unit 732, a stimulator unit 720, and an elongate lead 718. The internal receiver/transceiver unit 732 permits the cochlear implant system 710 to receive signals from and/or transmit signals to an external device 750. The external device 750 can be, for example, an off-the-ear (OTE) sound processor configured to be worn on the head that includes a receiver/transceiver coil 730 and sound processing components. Alternatively, the external device 750 can be just a transmitter/transceiver coil in communication with a behind-the-ear (BTE) sound processor that includes the sound processing components and microphone.

The implantable component 744 includes an internal coil 736, and preferably, an implanted magnet fixed relative to the internal coil 736. The magnet can be embedded in a pliable silicone or other biocompatible encapsulating material along with the internal coil 736. Signals sent generally correspond to external sound 713. The internal receiver/transceiver unit 732 and the stimulator unit 720 are hermetically sealed within a biocompatible housing, sometimes collectively referred to as a stimulator/receiver unit. Included magnets can facilitate the operational alignment of an external coil 730 and the internal coil 736 (e.g., via a magnetic connection), enabling the internal coil 736 to receive power and stimulation data from the external coil 730. The external coil 730 is contained within an external portion. The elongate lead 718 has a proximal end connected to the stimulator unit 720, and a distal end 746 implanted in a cochlea 740 of the user. The elongate lead 718 extends from stimulator unit 720 to the cochlea 740 through a mastoid bone 719 of the user. The elongate lead 718 is used to provide electrical stimulation to the cochlea 740 based on the stimulation data. The stimulation data can be created based on the external sound 713 using the sound processing components and based on sensory prosthesis settings.

In certain examples, the external coil 730 transmits electrical signals (e.g., power and stimulation data) to the internal coil 736 via a radio frequency (RF) link. The internal coil 736 is typically a wire antenna coil having multiple turns of electrically insulated single-strand or multi-strand platinum or gold wire. The electrical insulation of the internal coil 736 can be provided by a flexible silicone molding. Various types of energy transfer, such as infrared (IR), electromagnetic, capacitive and inductive transfer, can be used to transfer the power and/or data from external device to cochlear implant. While the above description has described internal and external coils being formed from insulated wire, in many cases, the internal and/or external coils can be implemented via electrically conductive traces.

FIG. 8 illustrates a retinal prosthesis system 800 that comprises an external device 810, a retinal prosthesis 801 and a mobile computing device 803. The retinal prosthesis system 800 can correspond to the sensory prosthesis 110. The retinal prosthesis 800 comprises a processing module 825 and a retinal prosthesis sensor-stimulator 890 is positioned proximate the retina 891 of a user. The external device 810 and the processing module 825 can both include transmission coils 856 aligned via respective magnet sets. Signals 851 can be transmitted using the coils 856.

In an example, sensory inputs (e.g., photons entering the eye) are absorbed by a microelectronic array of the sensor-stimulator 890 that is hybridized to a glass piece 892 including, for example, an embedded array of microwires. The glass can have a curved surface that conforms to the inner radius of the retina. The sensor-stimulator 890 can include a microelectronic imaging device that can be made of thin silicon containing integrated circuitry that convert the incident photons to an electronic charge.

The processing module 825 includes an image processor 823 that is in signal communication with the sensor-stimulator 890 via, for example, a lead 888 which extends through surgical incision 889 formed in the eye wall. In other examples, processing module 825 is in wireless communication with the sensor-stimulator 890. The image processor 823 processes the input into the sensor-stimulator 890, and provides control signals back to the sensor-stimulator 890 so the device can provide an output to the optic nerve. That said, in an alternate example, the processing is executed by a component proximate to, or integrated with, the sensor-stimulator 890. The electric charge resulting from the conversion of the incident photons is converted to a proportional amount of electronic current which is input to a nearby retinal cell layer. The cells fire and a signal is sent to the optic nerve, thus inducing a sight perception.

The processing module 825 can be implanted in the user and function by communicating with the external device 810, such as a behind-the-ear unit, a pair of eyeglasses, etc. The external device 810 can include an external light/image capture device (e.g., located in/on a behind-the-ear device or a pair of glasses, etc.), while, as noted above, in some examples, the sensor-stimulator 890 captures light/images, which sensor-stimulator is implanted in the user.

FIG. 9 illustrates an example of a suitable computing system 900 with which one or more of the disclosed examples can be implemented. Computing systems, environments, or configurations that suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The computing system 900 can be a single virtual or physical device operating in a networked environment over communication links to one or more remote devices. In examples, the sensory prosthesis 110, the user computing device 120, the clinician computing device 130, and the fitting server 140 can include one or more components or variations of components of the computing system 900.

In its most basic configuration, computing system 900 includes memory 993 and one or more processors 996. In the illustrated example, the system 900 further includes a network adapter 992, one or more input devices 998, and one or more output devices 999. The system 900 can include other components, such as a system bus, component interfaces, a graphics system, a power source (e.g., a battery), among other components.

The memory 993 is one or more software- or hardware-based computer-readable storage media operable to store information accessible by the one or more processors 996. The memory 993 can store, among other things, instructions executable by the one or more processors 996 to implement applications or cause performance of operations described herein, as well as other data. The memory 993 can be volatile memory (e.g., RAM), non-volatile memory (e.g., ROM), or combinations thereof. The memory 993 can include transitory memory or non-transitory memory. The memory 993 can also include one or more removable or non-removable storage devices. In examples, the memory 993 can include RAM, ROM, EEPROM (Electronically-Erasable Programmable Read-Only Memory), flash memory, optical disc storage, magnetic storage, solid state storage, or any other memory media usable to store information for later access. In examples, the memory 993 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 993 can include wired media such as a wired network or direct-wired connection, and wireless media such as acoustic, RF, infrared and other wireless media or combinations thereof.

The one or more processors 996 include one or more hardware or software processors, such as microprocessors, central processing units, etc. In many examples, the one or more processors 996 are configured to obtain and execute instructions, such as instructions stored in the memory 993. The one or more processors 996 can communicate with and control the performance of other components of the computing system 900.

The network adapter 992 is a component of the computing system 900 that provides network access over a network 902. The network adapter 992 can provide wired or wireless network access and can support one or more of a variety of communication technologies and protocols, such as ETHERNET, cellular, BLUETOOTH, near-field communication, and RF (Radiofrequency), among others. The network adapter 992 can include one or more antennas and associated components configured for wireless communication according to one or more wireless communication technologies and protocols.

The one or more input devices 998 are devices over which the computing system 900 receives input from a user. The one or more input devices 998 can include physically-actuatable user-interface elements (e.g., buttons, switches, or dials), touch screens, keyboards, mice, pens, and voice input devices, among others input devices.

The one or more output devices 999 are devices by which the computing system 900 can provide output to a user. The output devices 999 can include, displays, speakers, and printers, among other output devices.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
performing one or more first loudness scaling tests during which sounds are delivered to an ear of a user via a hearing device;
during the one or more first loudness scaling tests, providing the user with a first set of response options;
receiving, via the first set of response options, indications of the user's perceived loudness of the sounds delivered to the user during the one or more first loudness scaling tests; and
based on the indications of the user's perceived loudness of the sounds delivered to the user during the one or more first loudness scaling tests, adapting the first set of response options to a second set of response options for use during one or more second loudness scaling tests.

2. The method of claim 1, wherein adapting the first set of response options to a second set of response options for use during one or more second loudness scaling tests comprises:
increasing a level of precision in possible response options from a first precision level provided by the first set of response options to a second precision level provided by the second set of response options.

3. The method of claim 1, wherein adapting the first set of response options to a second set of response options for use during one or more second loudness scaling tests comprises:
decreasing a level of precision in possible response options from a first precision level provided by the first set of response options to a second precision level provided by the second set of response options.

4. The method of claim 1, wherein the first set of response options include a first number of possible responses and the second set of response options include a second number of possible responses, wherein the second number of possible responses is larger than the first number of possible responses.

5. The method of claim 1, wherein the first set of response options include a first number of possible responses and the second set of response options include a second number of possible responses, wherein the second number of possible responses is smaller than the first number of possible responses.

6. The method of claim 1, wherein the one or more second loudness scaling tests are the same as the one or more first loudness scaling tests.

7. The method of claim 1, wherein the one or more second loudness scaling tests include a greater level of loudness precision than a level of loudness precision included in the one or more first loudness scaling tests.

8. The method of claim 7, further comprising:
adapting the level of loudness precision included in the one or more first loudness scaling tests to the greater level of loudness precision included in the one or more second loudness scaling tests based on the indications of the user's perceived loudness of the sounds delivered to the user during the one or more first loudness tests.

9. The method of claim 1, wherein the one or more second loudness scaling tests include a greater level of frequency precision than a level of frequency precision included in the one or more first loudness scaling tests.

10. The method of claim 9, further comprising:
adapting the level of frequency precision included in the one or more first loudness scaling tests to the greater level of frequency precision included in the one or more second loudness scaling tests based on the indications of the user's perceived loudness of the sounds delivered to the user during the one or more first loudness tests.

11. The method of claim 1, wherein providing the user with the first set of response options comprises:
displaying the first set of response options at a display screen of a computing device.

12. The method of claim 1, further comprising:
setting one or more threshold levels or one or more comfort levels associated with the user's use of the hearing device based on the indications of the user's perceived loudness of the sounds delivered to the user during the one or more first loudness scaling tests.

13. The method of claim 1, further comprising:
setting one or more loudness growth functions associated with the user's use of the hearing device based on the indications of the user's perceived loudness of the sounds delivered to the user during the one or more first loudness scaling tests.

14. The method of claim 13, further comprising:
performing the one or more second loudness scaling tests during which sounds are delivered to the ear of the user via the hearing device;
during the one or more second loudness scaling tests, providing the user with the second set of response options; and
receiving, via the second set of response options, indications of the user's perceived loudness of the sounds delivered to the user during the one or more second loudness scaling tests.

15. The method of claim 14, further comprising:
determining one or more threshold levels or one or more comfort levels associated with the user's use of the hearing device based on the indications of the user's perceived loudness of the sounds delivered to the user during the one or more first loudness scaling tests; and
adjusting the one or more threshold levels or one or more comfort levels associated with the user's use of the hearing device based on the indications of the user's perceived loudness of the sounds delivered to the user during the one or more second loudness scaling tests.

16. The method of claim 14, further comprising:
determining one or more loudness growth functions associated with the user's use of the hearing device based on the indications of the user's perceived loudness of the sounds delivered to the user during the one or more first loudness scaling tests; and
adjusting the or more loudness growth functions associated with the user's use of the hearing device based on the indications of the user's perceived loudness of the sounds delivered to the user during the one or more second loudness scaling tests.

17. One or more non-transitory computer readable storage media comprising instructions that, when executed by at least one processor, are operable to:
display, via a display screen, a first set of loudness indicators;
perform one or more loudness scaling tests during which sounds are delivered to a user of a hearing device;
obtain results of the one or more loudness scaling tests via the first set of loudness indicators; and
based on the results of the one or more loudness scaling tests, adapt the first set of loudness indicators to a second set of loudness indicators for use in at least one additional loudness scaling test.

18. The one or more non-transitory computer readable storage media of claim 17, wherein the second set of loudness indicators provide a greater level of precision in a perceived loudness than a level of precision in a perceived loudness provided by the first set of loudness indicators.

19. The one or more non-transitory computer readable storage media of claim 17, wherein the second set of loudness indicators provide a reduced level of precision in a perceived loudness than a level of precision in a perceived loudness provided by the first set of loudness indicators.

20. The one or more non-transitory computer readable storage media of claim 17, wherein the first set of loudness indicators include a first number of loudness indicators and the second set of loudness indicators include a second number of loudness indicators, wherein the second number of loudness indicators is larger than the first number of loudness indicators.

21. The one or more non-transitory computer readable storage media of claim 17, wherein the first set of loudness indicators include a first number of possible indicators and the second set of loudness indicators include a second number of possible indicators, wherein the second number of loudness indicators is smaller than the first number of possible indicators.

22. The one or more non-transitory computer readable storage media of claim 17, wherein the one or more loudness scaling tests are performed using a first level of loudness precision and the at least one additional loudness scaling test is performed using a second level of loudness precision that is substantially the same as the first level of loudness precision.

23. The one or more non-transitory computer readable storage media of claim 17, wherein the one or more loudness scaling tests are performed using a first level of loudness precision and the at least one additional loudness scaling test is performed using a second level of loudness precision that is greater than the first level of loudness precision.

24. The one or more non-transitory computer readable storage media of claim 23, further comprising instructions operable to:
adapt the first level of loudness precision to the second level of loudness precision based on the results of the one or more loudness scaling tests.

25. The one or more non-transitory computer readable storage media of claim 17, wherein the one or more loudness scaling tests are performed using a first level of frequency precision and the at least one additional loudness scaling test is performed using a second level of frequency precision that is substantially the same as the first level of frequency precision.

26. The one or more non-transitory computer readable storage media of claim 17, wherein the one or more loudness scaling tests are performed using a first level of frequency precision and the at least one additional loudness scaling test is performed using a second level of frequency precision that is greater than the first level of frequency precision.

27. The one or more non-transitory computer readable storage media of claim 26, further comprising instructions operable to:
adapt the first level of frequency precision to the second level of frequency precision based on the results of the one or more loudness scaling tests.

28. The one or more non-transitory computer readable storage media of claim 17, further comprising instructions operable to:
determine one or more threshold levels or one or more comfort levels associated with the user's use of the hearing device based on the results of the one or more loudness scaling tests.

29. The one or more non-transitory computer readable storage media of claim 17, further comprising instructions operable to:
display, via a display screen, the second set of loudness indicators;
perform the at least one additional loudness scaling test during which sounds are delivered to the user of the hearing device; and
obtain results of the at least one additional loudness scaling test via the second set of loudness indicators.

30. The one or more non-transitory computer readable storage media of claim 29, further comprising:
determine one or more threshold levels or one or more comfort levels associated with the user's use of the hearing device based on the results of the one or more loudness scaling tests; and
adjust the one or more threshold levels or one or more comfort levels associated with the user's use of the hearing device based on the results of the at least one additional loudness scaling test.

31-44. (canceled)
